(19) United States
(12) Patent Application Publication
(10) Pub. No.: US 2023/0264191 A1
(43) Pub. Date: Aug. 24, 2023

(54) SYSTEMS AND METHODS FOR FABRICATING PHYSIOLOGICALLY RELEVANT IN VITRO VESSELS

(71) Applicant: The Texas A&M University System, College Station, TX (US)

(72) Inventors: Abhishek Jain, College Station, TX (US); Amirali Selahi, College Station, TX (US)

(73) Assignee: The Texas A&M University System, College Station, TX (US)

(21) Appl. No.: 18/112,279

(22) Filed: Feb. 21, 2023

Related U.S. Application Data
(60) Provisional application No. 63/312,177, filed on Feb. 21, 2022.

(51) Int. Cl. B01L 3/00; C12N 5/071

(52) U.S. Cl. CPC B01L 3/502707; B01L 3/502761; C12N 5/0602; B01L 3/502715; B01L 2300/0816; B01L 2400/0487; B01L 2300/0864; B01L 2200/025; B01L 2200/10

(57) ABSTRACT

A method for fabricating an in vitro vessel includes forming a substrate that defines a microfluidic passage therein extending along a longitudinal axis and defined by an inner surface, positioning the substrate in a vertical orientation whereby an acute angle is formed between the longitudinal axis of the microfluidic passage and the direction of gravity, and culturing a plurality of first cells in the microfluidic passage while the substrate is disposed in the vertical orientation whereby an annular layer of the plurality of first cells is formed in the microfluidic channel, wherein the layer of the plurality of first cells defines a lumen extending longitudinally through the microfluidic channel.

### CROSS-REFERENCE TO RELATED APPLICATIONS

This application claims benefit of U.S. provisional patent application Ser. No. 63/312,177 filed Feb. 21, 2022, and entitled “Systems and Methods for Fabricating Physiologically Relevant In Vitro Vessels Having a Cylindrical Vascular Structure,” which is hereby incorporated herein by reference in its entirety for all purposes.

### STATEMENT REGARDING FEDERALLY SPONSORED RESEARCH OR DEVELOPMENT

Not applicable.

### BACKGROUND

In vitro vessels intended to model in vivo human vessels are fabricated for a variety of purposes including, for example, identifying the mechanisms which drive vascular disease progression from a molecular level to an organ level. The success of such purposes, including the determination of the mechanisms which drive disease progression from the molecular level, is contingent on the physiological relevancy of the in vitro vessel. As one example, blood and lymphatic vascular functions of in vivo vessels are typically dependent on the radial and circumferential stresses and strains applied to the cells of the vessel due to the elliptical lumen of the in vivo vessel.

### BRIEF SUMMARY OF THE DISCLOSURE

An embodiment of a method for fabricating an in vitro vessel comprises (a) forming a substrate that defines a microfluidic passage therein extending along a longitudinal axis and defined by an inner surface, (b) positioning the substrate in a vertical orientation whereby an acute angle is formed between the longitudinal axis of the microfluidic passage and the direction of gravity, and (c) culturing a plurality of first cells in the microfluidic passage while the substrate is disposed in the vertical orientation whereby an annular layer of the plurality of first cells is formed in the microfluidic channel, wherein the layer of the plurality of first cells defines a lumen extending longitudinally through the microfluidic channel. In some embodiments, the plurality of first cells comprise at least one of lymphatic endothelial cells (LECs), vascular endothelial cells (VECs), lymphatic muscle cells (LMCs), and vascular muscle cells (VMCs). In some embodiments, the method comprises (d) culturing a plurality of second cells that are different from plurality of first cells in the microfluidic passage whereby an annular layer of the plurality of second cells is formed in the microfluidic channel. In certain embodiments, the layer of the plurality of first cells forms an annular inner layer of the first cells in the microfluidic channel and the layer of the plurality of second cells forms an outer layer of the plurality of second cells in the microfluidic channel that is radially positioned between the inner layer and the inner surface of the microfluidic channel. In certain embodiments, (d) is performed prior to (b). In some embodiments, the lumen has an elliptical cross-section.

An embodiment of an in vitro vessel comprises a substrate forming a microfluidic channel extending along a longitudinal axis and defined by an inner surface, wherein the microfluidic channel extends between a fluid inlet at a first end of the microfluidic channel and a fluid outlet located at a longitudinally opposed second end of the microfluidic channel, an annular outer layer of muscle cells positioned in the microfluidic channel and extending entirely around the longitudinal axis of the microfluidic channel, and an annular inner layer of endothelial cells positioned in the outer layer of muscle cells within the microfluidic channel and extending entirely around the longitudinal axis of the microfluidic channel, wherein the inner layer of endothelial cells defines a lumen extending longitudinally through the microfluidic channel and in fluid communication with both the fluid inlet and the fluid outlet formed in the substrate. In some embodiments, the muscle cells of the outer layer of muscle cells comprise lymphatic muscle cells (LMCs) and the endothelial cells of the inner layer of endothelial cells comprise lymphatic endothelial cells (LECs). In some embodiments, the muscle cells of the outer layer of muscle cells comprise vascular muscle cells (VMCs) and the endothelial cells of the inner layer of endothelial cells comprise vascular endothelial cells (VECs). In certain embodiments, the outer layer of muscle cells is embedded in an annular extracellular matrix (ECM) positioned in the microfluidic channel and containing collagen. In certain embodiments, a majority of the muscle cells comprising the outer layer of muscle cells are aligned substantially perpendicular to the longitudinal axis of the microfluidic channel. In some embodiments, a majority of the endothelial cells comprising the inner layer of endothelial cells are aligned substantially parallel to the longitudinal axis of the microfluidic channel. In some embodiments, a majority of the endothelial cells comprising the inner layer of endothelial cells are aligned substantially perpendicular to a majority of the muscle cells comprising the outer layer of muscle cells. In certain embodiments, the lumen has an elliptical cross-section.

An embodiment of an in vitro vessel comprises a substrate forming a microfluidic channel extending along a longitudinal axis and defined by an inner surface, wherein the microfluidic channel extends between a fluid inlet at a first end of the microfluidic channel and a fluid outlet located at a longitudinally opposed second end of the microfluidic channel, and an annular layer of endothelial cells positioned within the microfluidic channel and extending entirely around the longitudinal axis of the microfluidic channel, wherein the layer of endothelial cells defines a lumen having an elliptical cross-section and extending longitudinally through the microfluidic channel and in fluid communication with both the fluid inlet and the fluid outlet formed in the substrate. In some embodiments, the endothelial cells of the layer of endothelial cells comprise lymphatic endothelial cells (LECs). In some embodiments, the endothelial cells of the layer of endothelial cells comprise vascular endothelial cells (VECs). In certain embodiments, the elliptical cross-section of the lumen is defined by a major axis and a minor axis extending orthogonal to the major axis of the elliptical cross-section of the lumen, and wherein a ratio of the major axis to the minor axis is between 1.1:1 and 5:1. In certain embodiments, the elliptical cross-section of the lumen is defined by a major axis and a minor axis extending orthogonal to the major axis of the elliptical cross-section of the lumen, and wherein the ratio of the major axis to the minor axis is between 1:1 and 3:1. In some embodiments, a ratio of the minimum radial thickness of the layer of endothelial cells to the maximum radial thickness of the layer of endothelial cells is between 0.5:1 and 2:1. In some embodiments, the layer of endothelial cells positioned within the microfluidic channel defines an annular inner layer of endothelial cells, and an annular outer layer of muscle cells positioned in the microfluidic channel radially between the inner layer of endothelial cells and the inner surface of the microfluidic channel, and wherein the outer layer of muscle cells extends entirely around the longitudinal axis of the microfluidic channel. In certain embodiments, a ratio of the minimum radial thickness of the layer of muscle cells to the maximum radial thickness of the layer of muscle cells is between 0.5:1 and 2:1.

### BRIEF DESCRIPTION OF THE DRAWINGS

For a detailed description of exemplary embodiments of the disclosure, reference will now be made to the accompanying drawings in which:

FIG. 1 is a schematic view of an embodiment of an embodiment of a physiologically relevant in vitro vessel in accordance with principles described herein;

FIG. 2 is an enlarged cross-sectional view of an embodiment of a microfluidic channel of the in vitro vessel of FIG. 1 in accordance with principles described herein;

FIG. 3 is a flowchart of an embodiment of a method for fabricating a physiologically relevant in vitro vessel in accordance with principles described herein;

FIG. 4 is a schematic view of the in vitro vessel of FIG. 1;

FIGS. 5 and 6 are schematic views of an experimental lymphangion-chip in accordance with principles described herein;

FIG. 7 is a schematic illustrating the effect of gravity on lumen symmetry of the lymphangion-chip of FIGS. 5 and 6;

FIG. 8 is a graph illustrating lumen diameter as a function of hydrostatic pressure;

FIG. 9 is a graph illustrating lumen diameter as a function of collagen;

FIGS. 10 and 11 are graphs illustrating confluency as a function of time;

FIG. 12 is a graph illustrating gap width as a function of time;

FIG. 13 is a graph illustrating lymphatic endothelial cell (LEC) density as a function of time;

FIG. 14 is a graph illustrating LEC size as a function of time;

FIG. 15 is a graph illustrating lymphatic muscle cell (LMC) density as a function of time;

FIG. 16 is a schematic of a microfluidic channel of the lymphangion-chip of FIGS. 5 and 6;

FIG. 17 is a chart illustrating the relative orientation of LECs and LMCs under different shear conditions;

FIG. 18 is a heatmap indicating LEC mean orientation angle in both monoculture and coculture configurations while being exposed to different shear conditions;

FIG. 19 is a heatmap indicating LMC mean orientation angle in both monoculture and coculture configurations while being exposed to different shear conditions;

FIG. 20 is a graph illustrating normalized fluorescence intensity as a function of distance;

FIG. 21 is a graph illustrating measured permeability for the lymphangion-chip of FIGS. 5 and 6; and

FIG. 22 is a graph illustrating permeability of the lymphangion-chip of FIGS. 5 and 6.

### DETAILED DESCRIPTION OF THE DISCLOSED EMBODIMENTS

The following discussion is directed to various exemplary embodiments. However, one skilled in the art will understand that the examples disclosed herein have broad application, and that the discussion of any embodiment is meant only to be exemplary of that embodiment, and not intended to suggest that the scope of the disclosure, including the claims, is limited to that embodiment.

Certain terms are used throughout the following description and claims to refer to particular features or components. As one skilled in the art will appreciate, different persons may refer to the same feature or component by different names. This document does not intend to distinguish between components or features that differ in name but not function. The drawing figures are not necessarily to scale. Certain features and components herein may be shown exaggerated in scale or in somewhat schematic form and some details of conventional elements may not be shown in interest of clarity and conciseness.

In the following discussion and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct connection, or through an indirect connection via other devices, components, and connections. In addition, as used herein, the terms “axial” and “axially” generally mean along or parallel to a central axis (e.g., central axis of a body or a port), while the terms “radial” and “radially” generally mean perpendicular to the central axis. For instance, an axial distance refers to a distance measured along or parallel to the central axis, and a radial distance means a distance measured perpendicular to the central axis. Further, as used herein, the terms “approximately,” “about,” “substantially,” and the like mean within 10% (i.e., plus or minus 10%) of the recited value. Thus, for example, a recited angle of “about 80 degrees” refers to an angle ranging from 72 degrees to 88 degrees.

As described above, the success of in vitro vessels (blood and lymphatic) in modeling in vivo human vessels is contingent on the physiological relevancy of the in vitro vessel such as, for example, the radial and circumferential stresses and strains applied to the cells of the vessel due to the elliptical lumen of the in vivo vessel. Indeed, conventional techniques for fabricating in vitro vessels have generally been limited in providing vessels having a physiologically relevant lumen, and thus are typically limited in their ability to successfully model in vitro human vessels.

Specifically, conventionally fabricated vessels typically comprise an extracellular matrix (ECM) formed in a microfluidic channel thereof that is not uniformly dispersed about the circumference of the channel, and instead is asymmetrical being thick at the bottom of the channel and thin at the opposing top of the channel. Additionally, conventionally fabricated vessels are typically monocultured with a single cell type and thus do not include both the mural and endothelial cells found in in vivo vessels. The limitations of conventional in vitro vessels may arise due to the viscous finger patterning method used to fabricate conventional in vitro vessels in which viscous fingering is relied on in which a less viscous fluid (e.g., the cell medium) displaces a more viscous liquid (e.g., liquid collagen) in the microfluidic channel of the in vitro vessel, thereby creating finger-shaped structures therein. This technique does not account for or control fluid dynamics such as fluid pressure head across the fluid inlet and outlet of the microfluidic channel as well as gravity effects which influence the configuration of the lumen formed in the microfluidic channel.

As a result of the limitations of the viscous fingering technique, conventional in vitro vessels typically lack a cylindrical lumen which either does not include mural or parenchymal cells (e.g., smooth muscle cells of the vessel), or does not have a uniformly thick layer of muscle cells extending along the circumference of the lumen. For instance, in conventional techniques, in vitro vessels are typically culture in an orientation where the force of gravity acts on the cultured muscle cells in a direction perpendicular to a longitudinal axis of the vessel. This perpendicularly oriented gravitational force draws muscle cells located towards a top of the lumen towards the bottom of the lumen as the cells are cultured within the in vitro vessel, resulting in a relatively thin layer of muscle cells at the top of the lumen and a relatively thick layer of muscle cells at the bottom of the lumen. This lack of uniformity, among other things, inhibits the formed in vitro vessel from mimicking the true functionality of in vivo vessels.

This disclosure relates to systems and methods for fabricating physiologically relevant in vitro vessels having an elliptical lumen. Specifically, this disclosure describes embodiments of in vitro vessels having elliptical lumen in which include mural or parenchymal cells formed as a uniformly distributed layer along a circumference of the vessel. The vessel may comprise a blood vessel (e.g., a vein or artery) or lymphatic vessel. In addition to the uniformly distributed layer of muscle cells, embodiments of physiologically relevant in vitro vessels descried herein include a confluent layer of endothelial cells surrounded by the uniformly distributed layer of muscle cells. The vessel may also contain other cells or substances such as unhealthy cells (e.g., cancer cells) used to model disease progression in the in vitro vessel. In some embodiments, the in vitro vessels described herein are microfluidic in size having a diameter of 500 microns or less.

The physiologically relevant vessels described herein may be fabricated using a gravitational lumen patterning (GLP) technique for forming both a confluent layer of endothelial cells (intima) surrounded by the uniformly distributed muscle cells (media). Using the GLP technique, the vessel may be oriented such that the force of gravity acts on the cells cultured therein in a direction parallel to a longitudinal axis of the in vitro vessel. In this manner, the force of gravity does not draw cells from the top of the vessel towards the bottom of the in vitro vessel, and instead, the cells cultured (e.g., endothelial cells, muscle cells, etc.) in the vessel are permitted to achieve a uniform distribution along the circumference of the in vitro vessel.

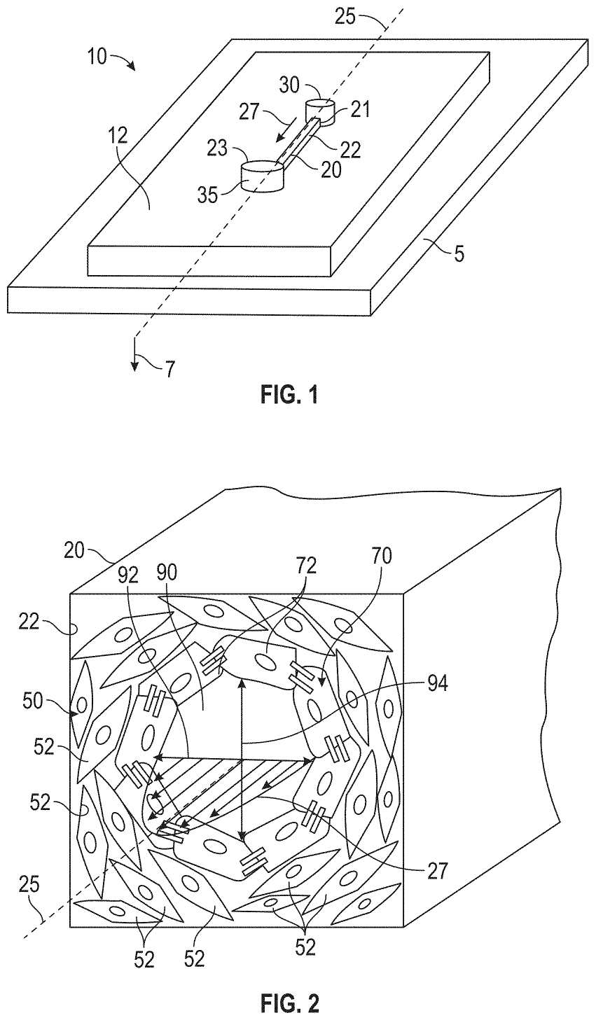

Referring now to FIG. 1, an embodiment of a physiologically relevant in vitro vessel 10 is shown. In vitro vessel 10 generally includes a substrate 12 positioned on a platform 5. In some embodiments, substrate 12 is formed using a soft lithography process in which the substrate 12 comprises polydimethylsiloxane (PDMS); however, it may be understood that substrate 12 may be fabricated in a variety of ways and may comprise a variety of different materials. Substrate 12 defines a microfluidic channel 20 therein that extends along a longitudinal or central axis 25 and is defined by an inner surface 22. Microfluidic channel 20 is shown oriented such that the longitudinal axis 25 of channel 20 extends orthogonal to the direction of gravity 7. However, as will be described further herein, the orientation of longitudinal axis 25 of microfluidic channel 20 relative to the direction of gravity 7 may be controlled during the fabrication of the in vitro vessel 10 as part of a gravitational lumen patterning (GLP) fabrication process.

In this exemplary embodiment, microfluidic channel 20 extends from a first or inlet end 21 to a second or outlet end 23 longitudinally opposite the inlet end 21. In some embodiments, the microfluidic channel 20 has a longitudinal length extending between the inlet end 21 and the outlet end 23 that is equal to or less than 10 millimeters (mm) in length (e.g., 5 mm). Additionally, in some embodiments, the microfluidic channel 20 has a width and a corresponding height that are each equal to or less than 1 mm (e.g., 0.9 mm). However, it may be understood that the size and geometry of microfluidic channel 20 may vary in other embodiments.

In addition to microfluidic channel 20, a fluid inlet 30 and a fluid outlet 35 are also formed in the substrate 12. Particularly, fluid inlet 30 is located adjacent the inlet end 21 of microfluidic channel 20 while the fluid outlet 35 is located adjacent the outlet end 23 of microfluidic channel 20. In this configuration, the microfluidic channel 20 is in fluid communication with both the fluid inlet 30 and the fluid outlet 35 whereby a fluid flow 27 may be established through the fluid inlet 30, into and through the microfluidic channel 20, and through the fluid outlet 35. For example, a syringe pump may be connected to the fluid inlet 30 for establishing the fluid flow 27 through the microfluidic channel 20 as desired by a user of the in vitro vessel 10.

Referring to FIG. 2, a cross-section of the microfluidic channel 20 of in vitro vessel 10 is shown. In this exemplary embodiment, an annular first or outer layer 50 is formed in the microfluidic channel 20 and extending longitudinally between the inlet end 21 and the outlet end 23 of the microfluidic channel 20. In this exemplary embodiment, the outer layer 50 extends continuously and entirely (360°) around the longitudinal axis 25 of microfluidic channel 20. Additionally, in this exemplary embodiment, an annular second or inner layer 70 is also formed in the microfluidic channel 20 and extending longitudinally between the inlet end 21 and the outlet end 23 of the microfluidic channel 20. The inner layer 70 defines a fluid passage or lumen 90 through which the fluid flow 27 extends and which is in fluid communication with both the fluid inlet 30 and the fluid outlet 35 formed in the substrate 12. In this configuration, lumen 90 extends longitudinally through the microfluidic channel 20 such that the longitudinal axis 25 extends longitudinally through lumen 990.

Additionally, in this exemplary embodiment, the inner layer 70 extends continuously and entirely (360°) around the longitudinal axis 25 of microfluidic channel 20. In this configuration, the outer layer 50 is positioned radially between the inner layer 70 and the inner surface 22 of microfluidic channel 20 whereby the outer layer 50 entirely surrounds or encircles the inner layer 70 positioned therein. While in this exemplary embodiment the in vitro vessel 10 includes both an outer layer 50 and an inner layer 70, it may be understood that in other embodiments the in vitro vessel 10 may include only a single layer (e.g., one of layers 50 and 70) or more than two separate layers (e.g., layers in addition to layers 50 and 70) within the microfluidic channel 20 thereof.

In this exemplary embodiment, the lumen 90 formed by the inner layer 70 has an elliptical cross-section defined by a first or major axis 92 and a second or minor axis 94 extending orthogonal to the major axis 92, and where each axis 92 and 94 intersects the longitudinal axis 25 of microfluidic channel 20. The elliptical cross-section of lumen 90 is more consistent to in vivo vessels, such as human lymphatic and vascular vessels, than the lumen formed by conventional in vitro vessels which may instead have a rectangular or square cross-section. Particularly, the elliptical geometry of lumen 90 permits the in vitro vessel 10 to better mimic the physiological conditions (e.g., physiological stress and strain conditions) found in in vivo vessels. It may be understood that elliptical geometry of lumen 90 may vary between embodiments based on the needs of the particular application. For example, in some embodiments, the ratio of the major axis 92 to the minor axis 94 of lumen 90 ranges approximately between 1:1 (providing a circular cross-section) and 5:1. In some embodiments, the ratio of the major axis 92 to the minor axis 94 of lumen 90 ranges approximately between 1.1:1 and 3:1. In certain embodiments, the ratio of the major axis 92 to the minor axis 94 of lumen 90 ranges approximately between 1.5:1 and 2.5:1. Additionally, in some embodiments, the lumen 90 ranges approximately from 10 microns to 800 microns in diameter.

In this exemplary embodiment, the outer layer 50 formed in microfluidic channel 20 comprises a plurality of first cells 52 (only some of which are labeled in FIG. 2 for clarity) while the inner layer 70 formed in the microfluidic channel 20 and surrounded by the outer layer 50 comprises a plurality of second cells 72 (again, only some of which are labeled in FIG. 2 for clarity) which are different from the first cells 52. The second cells 72 of inner layer 70 may be separately cultured in the microfluidic channel 20 from the first cells 52. Particularly, first cells 52 and second cells 72 may be cultured using a GLP fabrication process as will be discussed further herein whereby the first cells 52 and second cells 72 are each distributed substantially uniformly circumferentially about the longitudinal axis 25 of microfluidic channel 20 such that the cells 52 and/or 72 do not collect or congregate at one circumferential location along the circumference of the microfluidic channel 20 (e.g., towards the vertical bottom of the microfluidic channel 20). In this manner, while the radial thickness of each layer 50 and 70 may vary to some degree moving circumferentially along the layer 50 and 70, the variation in radial thickness of each layer 50 and 70 moving circumferentially along the layer 50 and 70 is not particularly significant. For instance, the thickness For example, in some embodiments, the ratio of the minimum radial thickness of each layer 50 and 70 to the maximum radial thickness of each layer 50 and 70 is approximately between 0.5:1 and 2:1. In certain embodiments, the ratio of the minimum radial thickness of each layer 50 and 70 to the maximum radial thickness of each layer 50 and 70 is approximately between 1:1 and 1.3:1. In some embodiments, the ratio of the minimum radial thickness of each layer 50 and 70 to the maximum radial thickness of each layer 50 and 70 is approximately between 1:1 and 1.2:1.

The first cells 52 and second cells 72 may each have a specific alignment relative to the direction of the longitudinal axis 25 of microfluidic channel 20. For example, in some embodiments, a majority of the first cells 52 comprising the outer layer 50 align in a direction that is substantially perpendicular to the longitudinal axis 25 of microfluidic channel 20 while a majority of the second cells 72 forming the inner layer 70 align in a direction that is substantially perpendicular to longitudinal axis 25. In this manner, the first cells 52 of outer layer 50 wrap circumferentially around the axially-aligned (substantially aligned with longitudinal axis 25) second cells 72 which align in the direction of fluid flow 27 through the lumen 90. Additionally, in this arrangement, a majority of the first cells 52 align in a direction that is substantially perpendicular to the direction in which a majority of the second cells 72 are aligned.

As described above, the first cells 52 are different from the second cells 72. Particularly, in this exemplary embodiment, the first cells 52 comprise mural or muscle cells while the second cells 72 comprise endothelial cells. Thus, first cells 52 may also be referred to herein as muscle cells 52 while second cells 72 may also be referred to herein as endothelial cells 72. In some embodiments, in vitro vessel 10 comprises a physiologically relevant lymphatic in vitro vessel with muscle cells 52 comprising LMCs and endothelial cells 72 comprising LECs. In other embodiments, in vitro vessel 10 comprises a physiologically relevant vascular or blood in vitro vessel with muscle cells 52 comprising vascular muscle cells (VMCs) and endothelial cells 72 comprising vascular endothelial cells (VECs). It may be understood that in other embodiments first cells 52 may comprise cells other than muscle cells and second cells 72 may comprise cells other than endothelial cells based on the requirements of the given application. For example, in some embodiments, first cells 52 or second cells 72 may comprise any type of perivascular cells such as, for example, pericytes, fibroblasts or resident immune cells, like macrophages.

In some embodiments, the outer layer 50 of in vitro vessel 10 comprises an ECM including the muscle cells 52 as well as other materials such as collagen hydrogel. Additionally, in some embodiments, in vitro vessel 10 may comprise a different number of muscle cells 52 compared to endothelial cells 72 located in the microfluidic channel 20. For example, in some embodiments, the ratio of the number of endothelial cells 72 to the number of muscle cells 52 ranges approximately between 1:3 and 3:1. However, it may be understood that this range may vary substantially depending on the given application.

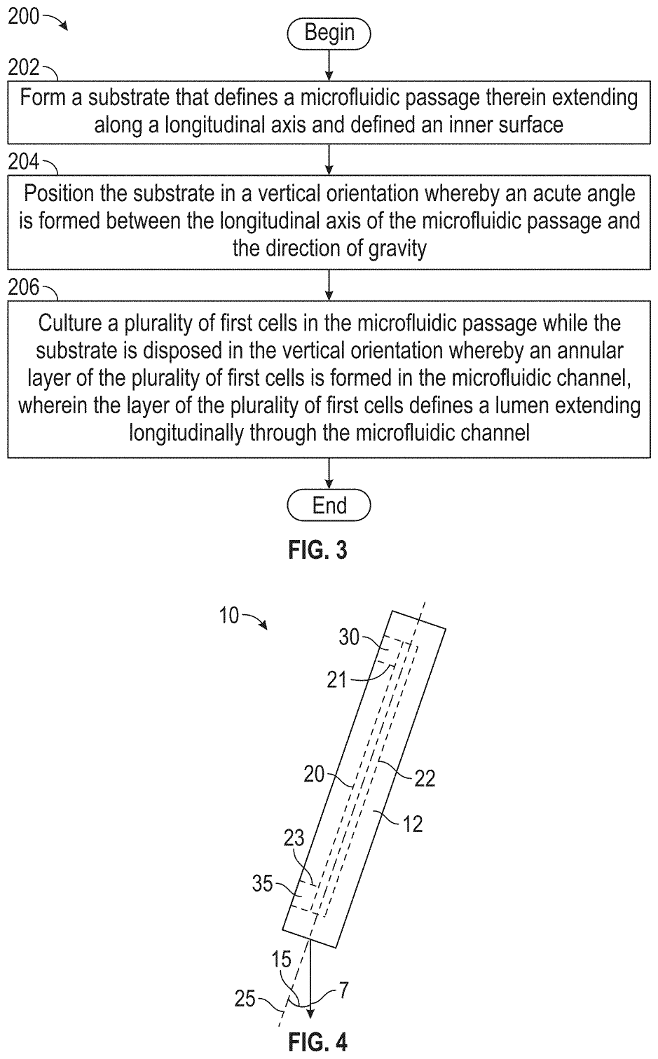

Referring now to FIG. 3, an embodiment of a method 200 for fabricating an invitro vessel (e.g., in vitro vessel 10 shown in FIGS. 1 and 2) is shown. Initially, at block 202 method 200 comprises forming a substrate that defines a microfluidic passage therein extending along a longitudinal axis and defined by an inner surface. In some embodiments, block 202 comprises forming the substrate 12 shown in FIG. 1 that defines the microfluidic passage 20 shown in FIGS. 1 and 2 extending therein along the longitudinal axis 25 and defined by the inner surface 22. In certain embodiments, the substrate (e.g., substrate 12) may be formed from PDMS using a soft lithography process; however, it may be understood the substrate may be formed from a variety of different materials using a variety of different fabrication techniques.

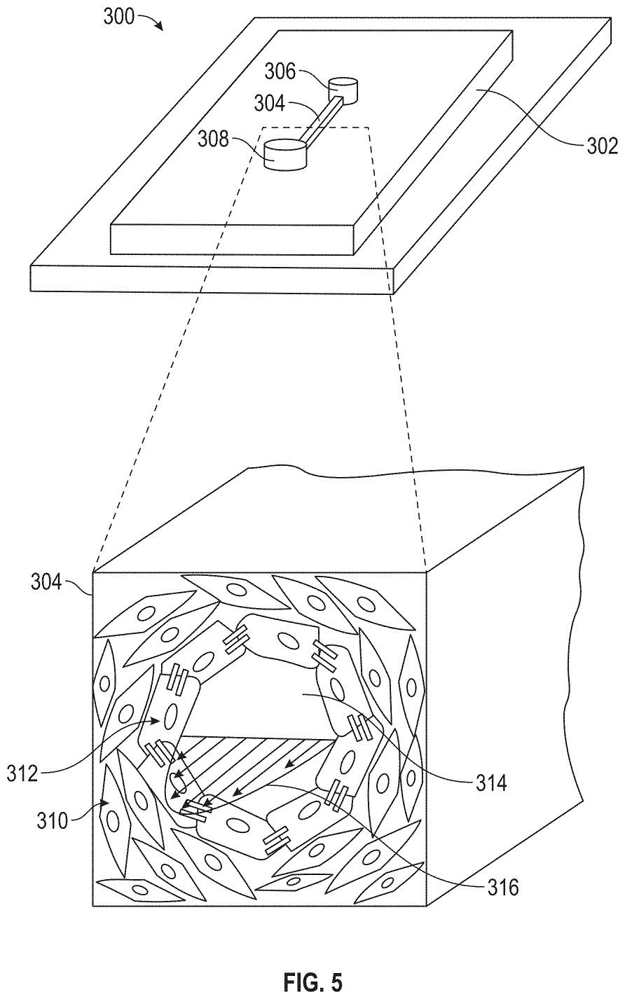

At block 204, method 200 comprises positioning the substrate in a vertical orientation whereby an acute angle is formed between the longitudinal axis of the microfluidic passage and the direction of gravity. In some embodiments, block 204 comprises positioning the substrate 12 shown in FIG. 1 in a vertical orientation whereby an acute angle is formed between the longitudinal axis 25 of the microfluidic passage 20 shown in FIGS. 1 and 2 and the direction of gravity. For example, referring briefly to FIG. 4, the substrate 12 of in vitro vessel 10 is shown in a vertical orientation whereby an acute angle 15 is formed between the longitudinal axis 25 of microfluidic channel 20 and the direction of gravity 7. In some embodiments, the acute angle 15 is equal to or less than 30°. In some embodiments, the acute angle 15 is equal to or less than 20°. In certain embodiments, the acute angle 15 is equal to or less than 15°. In certain embodiments, the acute angle 15 is equal to or less than 10°. In some embodiments, the acute angle 15 is equal to or less than 5°. Finally, in some embodiments, the acute angle 15 is equal to or less than 1°. It may also be understood that in some embodiments the acute angle 15 may be zero with the substrate 12 oriented perfectly vertical with respect to the direction of gravity 7.

Referring again to FIG. 3, at block 206, method 200 comprises culturing a plurality of first cells in the microfluidic passage while the substrate is disposed in the vertical orientation whereby an annular layer of the plurality of first cells is formed in the microfluidic channel, wherein the layer of the plurality of first cells defines a lumen extending longitudinally through the microfluidic channel. In some embodiments, block 206 comprises culturing one of the cells 52 and 72 shown in FIG. 2 in the microfluidic passage 20 of the substrate 12 shown in FIG. 1 while the substrate 12 is disposed in the vertical orientation (e.g., the orientation of substrate 12 shown in FIG. 4) whereby an annular layer (e.g., one of layers 50 and 70 shown in FIG. 2) are formed in the microfluidic channel 20.

While the inner layer 70 defines the lumen 90 in FIG. 2, it may be understood that in other embodiments in which outer layer 50 comprises the only cell-containing layer present in microfluidic channel 20 such that the outer layer 50 defines the lumen 90. In other words, the “first cells” of block 206 may correspond to the first cells 52 shown in FIG. 2, the second cells 72 shown in FIG. 2, or other cells different from both the first cells 52 and second cells 72. Thus, while the first cells of block 206 may comprise endothelium cells such as LECs and VECs, muscle cells such as LMCs and VMCs, and/or cells other than either endothelium cells or muscle cells.

In some embodiments, method 200 additionally includes culturing a plurality of second cells that are different from plurality of first cells in the microfluidic passage whereby an annular layer of the plurality of second cells is formed in the microfluidic channel. For example, the layer of second cells may comprise an annular outer layer formed in the microfluidic channel while the layer of first cells may comprise an annular inner layer formed in the microfluidic channel within the outer layer of second cells. In some embodiments, the outer layer of second cells may be formed in the microfluidic channel before the inner layer of first cells is formed in the microfluidic channel. For example, the outer layer may comprise a layer of muscle cells while the inner layer may comprise a layer of endothelial cells. The outer layer may be formed in the microfluidic channel with the substrate disposed in the vertical orientation or in an orientation that differs from the vertical orientation such as a horizontal orientation in which the longitudinal axis of the microfluidic channel extends orthogonally to the direction of gravity.

It may be understood that the vertical orientation of the substrate 12 and microfluidic channel 20 formed therein mitigates the buoyancy effects that might otherwise impair the formation of the elliptical lumen 90. By orienting the substrate 12 vertically, the microfluidic channel 20 may be oriented such that the force of gravity acts on the cells cultured therein in a direction parallel to a longitudinal axis 25 of the microfluidic channel 20. In this manner, the force of gravity does not draw cells from the top of the microfluidic channel 20 towards the bottom of the microfluidic channel 20, and instead, the cells cultured (e.g., endothelial cells, muscle cells, etc.) in the microfluidic channel 20 are permitted to achieve a uniform distribution along the circumference of the microfluidic channel 20.

Experiments were conducted regarding the fabrication of physiologically relevant in vitro vessels. It may be understood that the following experiments described herein are not intended to limit the scope of this disclosure and the embodiments described above and shown in FIGS. 1-4.

Referring now to FIG. 5, an experimental setup in the form of an in vitro vessel 300 is shown. Particularly, in vitro vessel 300 comprises a lymphangion-chip configured to mimic a human in vivo lymphangion which is the unit of lymphatic vessel located between two adjacent valves of the lymphatic vessel. Thus, in vitro vessel 300 may also be referred to herein as lymphangion-chip 300. As shown in FIG. 5, the lymphangion-chip 300 generally includes a platform 302 in which a microfluidic channel 304 is formed extending between a fluid inlet 306 and a fluid outlet 308. Additionally, a plurality of LMCs 310 and a plurality of LECs 312 are cultured onto an inner surface of the microfluidic channel 304 whereby both the LMCs 310 and LECs 312 extend circumferentially across the entirety of the periphery of the microfluidic channel 304 whereby a central fluid passage 314 is formed within the microfluidic channel 304 and which is surrounded by both the cultured LMCs 310 and LECs 312. A fluid flow 316 may be established through the fluid passage 314 between the inlet 306 and the outlet 308 of platform 302. The LMCs 310 and LECs 312 of lymphangions-chip 300 were cocultured within an ECM for several days under fluid shear. With a fabrication technique specifically developed to wrap LMCs 310 uniformly around the LECs 312, lymphangion-chip 300 illustrated that both LMCs 310 and LECs 312 maintain their essential phenotype, growth and subendothelial characteristics, and morphological alignment, as either seen or expected in vivo. Following fabrication of the lymphangion-chip 300, the sensitivity of cocultured LECs 312 and LMCs 310 due to shear and inflammatory cues were characterized. Overall, the data suggest that the lymphangion-chip 300 serves as an experimental model for preclinical lymphatic (and blood) vascular research and pharmacological testing.

Design and Engineering of the Lymphangion-Chip

The lymphangion-chip 300 was fabricated as a platform technology offering control over geometry, mechanical properties, and fluid dynamics relevant to the diversity of lymphangions in vivo. Importantly, the intent behind the fabrication of the lymphangion-chip 300 was to create a cylindrical/elliptical microfluidic organ-chip consisting of a monoculture of LECs 312 surrounded by multiple layers of uniformly thick LMCs 310 embedded inside collagen hydrogel as an initial supporting ECM. While several lumen forming techniques currently exist, conventional lumen forming techniques generally cannot be made cylindrical or elliptical while also supporting a culture of muscle cells in an in vivo like morphology. In particular, the conventional viscous finger patterning technique has now been adopted several times to make microfluidic endothelialized lumens, but in coculture settings, this technique has failed to demonstrate a uniform distribution of wrapped muscle cells around the endothelium. Generally, this technique is believed to rely primarily on viscous fingering (also known as Saffman-Taylor instability) that is a fluid dynamics phenomenon in which a less viscous fluid (cell medium) flows through and displaces a more viscous liquid (liquid collagen), creating finger-shaped structures. It was hypothesized as part of this experimental study that in this conventional technique, convective fluid dynamics characteristics—fluid pressure head across the inlet and outlet of the microfluidic channel and gravity—also determine the size and position of the lumens in addition to displacement due to the differences in the two fluids' viscosities.

To test this hypothesis, an LMC-hydrogel mixture was perfused into the lymphangion-chip 300 by attaching an inlet reservoir to the fluid inlet 306, and producing a vacuum inside the microfluidic channel 304 using a syringe connected to the outlet 308. This process was performed both when the lymphangion-chip 300 was positioned horizontally on an incubator rack (corresponding to the viscous fingering method) and when the lymphangion-chip 300 was rotated by approximately 90° from horizontal to align the longitudinal axis of the microfluidic channel with the gravitational direction (corresponding to gravitational lumen patterning (GLP)).

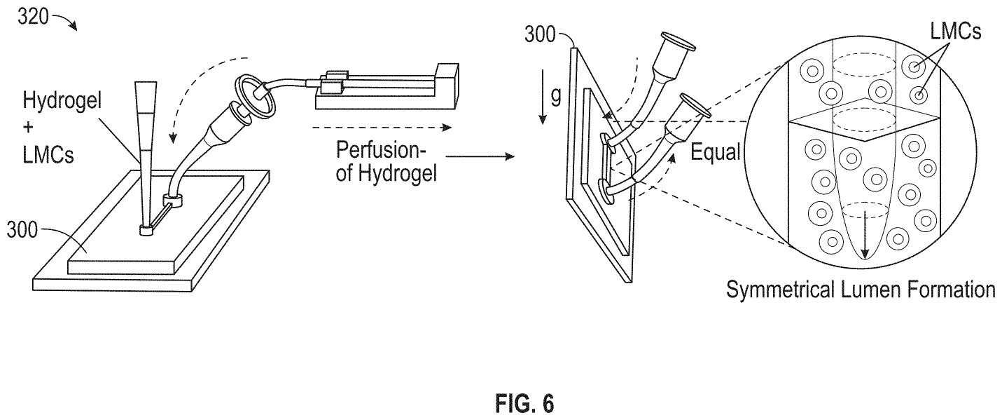

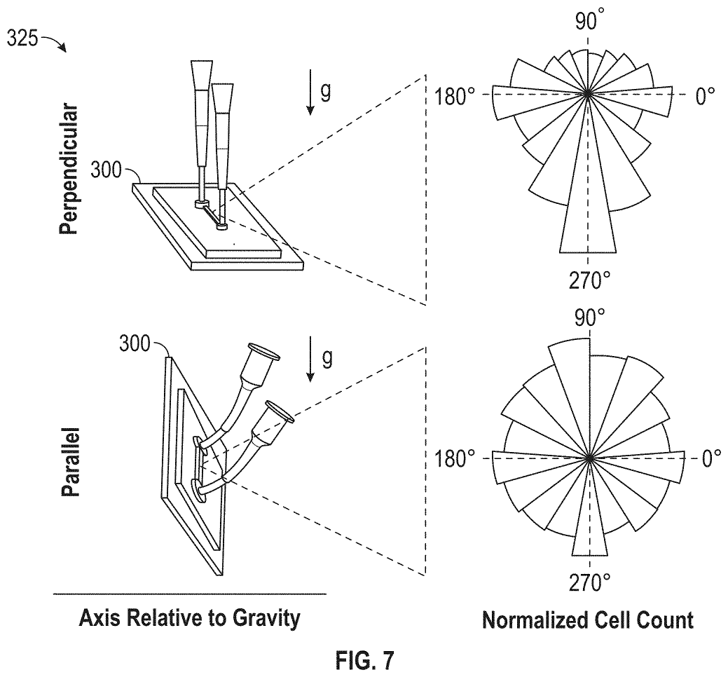

Referring to FIGS. 6 and 7, a schematic 320 is provided by FIG. 6 illustrates the perfusion of an LMC-matrix mixture into the lymphangion-chip 300 using a syringe connected to the fluid outlet 308. Schematic 320 also illustrates the lymphangion-chip 300 being rotated by 90° so that the microfluidic channel 304 aligned parallel to the direction of gravity. While keeping the lymphangion-chip 300 in the vertical position, a curved tip filled with LMC medium was added to the fluid inlet 306 while rotating the outlet tip so that both tips share a horizontal plane (equal level) to prevent the cell medium from flowing out of the lymphangion-chip 300. In this case, the less viscous fluid (cell medium) would wash away the highly viscous fluid (hydrogel) and form a 3D symmetrical lumen. Additionally, FIG. 7 illustrates a schematic 325 illustrating the effect of gravity on lumen symmetry when the axis of the microfluidic channel 304 is perpendicular and parallel to the direction of gravity.

While keeping the lymphangion-chip 300 in this position, the less viscous fluid (cell medium) displaced the highly viscous fluid (collagen-LMC mixture) and formed a lumen as indicated by schematic 320 of FIG. 6. When the GLP method was adopted, the thickness of the ECM was relatively conserved in different angular positions around the inner hollow cavity or fluid passage 314 (shown in FIG. 5), as observed by doping the matrix with fluorescent beads or by directly visualizing the LMCs within the matrix. This relatively uniform distribution was absent when the classical viscous fingering technique was adopted. The buoyancy effect may be responsible for this absence, where the higher density fluid (collagen) tended to displace the lower density liquid (cell medium) and push it upwards towards the top of the fluid passage 314, thus resulting in a variably thick ECM around the lumen. However, by maintaining the lymphangion-chip 300 vertically, thereby aligning the gravitational force in the axial direction of microfluidic channel 304, this transverse effect of buoyant force was prevented and instead a 3D lumen was established with a nearly symmetrical cross-section.

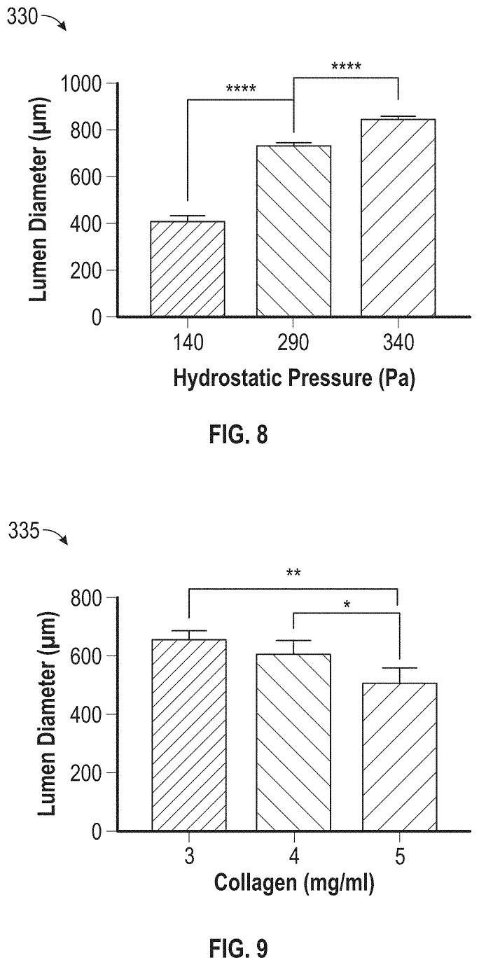

In addition to the experiments described above, as part of this experimental study, the GLP technique was employed to build lymphangion-chips 300 of variable sizes and ECM thicknesses. Particularly, by altering the hydrostatic pressure at the inlet 306 (e.g., 140 pascals (Pa) to 340 Pa) while performing the GLP technique, the lumen diameter could be modulated from a range of 400 microns (μm) to 800 μm (FIGS. 3A and B). Referring to FIGS. 8 and 9, graphs 330 and 335 are shown illustrating lumen diameter in units of μm as a function of Hydrostatic pressure in units of Pa (graph 330) and as a function of collagen in units of milligrams per milliliters (mg/ml) (graph 335). Given that the collagen concentration is directly proportional to its viscosity and hydraulic resistance, it was found that increasing the collagen concentration from 3 mg/ml to 5 mg/ml (˜60%) during GLP resulted in an approximately 30% lumen size reduction. The outer lumen diameter (i.e., the vessel's thickness) was also altered during this experimentation by manufacturing molds with 600 μm, 400 μm, and 200 μm channel widths and confirmed that lumen formation using GLP was successful in this range as well. Taken together, these results indicated that mechanical factors, such as the hydrostatic pressure, microchannel size, and gravitational effect, as well as our supporting biomaterial—collagen concentration—can be varied to create hollow lumens of a wide range of sizes, thicknesses, and interstitial mechanical properties relevant to lymphatic vessels.

Reconstitution of Lymphatic Endothelial and Muscle Tissues in the Lymphangion-Chip

Given that the endothelial and muscle cells are the two main tissue components of a lymphatic vessel, experimental studies were conducted to confirm the possibility of co-culturing these two lymphatic cell types in a lymphangion-chip 300 using just one cell culture medium formulation. Initially, only LECs were seeded on the luminal side of the chip which resulted in LECs forming a monolayer of confluent endothelial cells with properly formed cell-cell junctions in approximately two days. Using cell specific culture medium, LECs remained confluent even after approximately five days, and barrier integrity was maintained. In the next experiment, LMCs alone were mixed with collagen and perfused within a lymphangion-chip 300. After approximately five days of monoculture using the medium specified for this cell, LMCs formed multilayers of oval-shaped structures embedded inside the collagen matrix, similar to the observed in vivo morphology. The distribution and proliferation were evaluated and thereby confirmed the presence of LMCs all over the lumen.

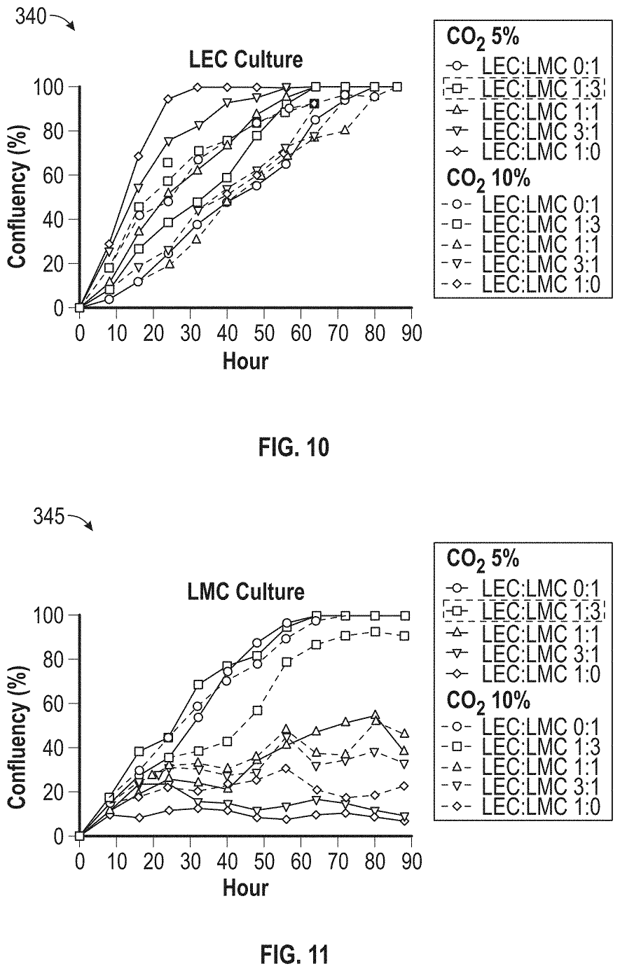

Next, to identify the standard cell culture conditions for a successful coculture, the effect of environmental CO2 percentage and cell medium combination on LMC and LEC growth, respectively, was investigated. Referring to FIGS. 10 and 11, graphs 340 and 345 are shown each illustrating confluency (%) as a function of time in hours, where graph 340 corresponds to LEC growth while graph 345 corresponds to LMC growth. It was observed that even though LECs reached full confluency in all combinations of LEC:LMC medium, a CO2 of 5% and LEC:LMC ratios of 1:0 and 3:1 resulted in 100% cell coverage in less than 60 hours. In contrast, LMCs were more sensitive to LEC:LMC medium for which only LEC:LMC ratios of 1:0 and 1:3 resulted in 100% confluency as indicated in graphs 340 and 345, respectively.

Based on these datasets, we identified that a CO2 of 5% and LEC:LMC medium ratio of 1:3 was identified as suitable for the coculture. Using the derived formulation, a mixture of LMCs and collagen was perfused through the lymphangion-chip 300, followed by lumen formation via the GLP technique. After approximately one day, LECs were seeded through the lumen on the collagen face and were kept in the incubator. Confocal fluorescence microscopy analysis revealed that a confluent layer of endothelium surrounded by multiple layers of muscle cells was formed in the lymphangion-chip 300. In lymphangion-chips 300 that has been cocultured, the average lumen inner diameter and muscle layer thickness was measured to be nearly 750 μm and 150 μm, respectively. These images provided the first evidence of a successful lymphatic vessel-on-chip consisting of both LECs and LMCs cultured together using a common medium formulation.

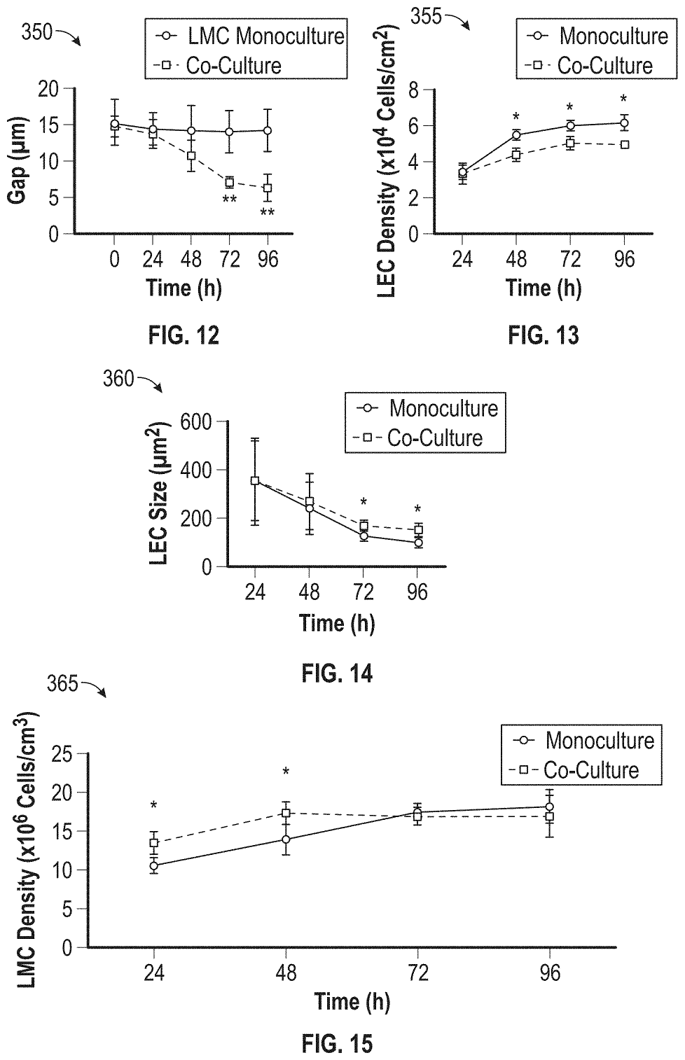

To assess the physiological relevance of the LEC and LMC interface, the on-chip endothelium and muscle layer gap as well as cell growth in coculture versus monoculture conditions were examined. Referring to FIGS. 12-15, graphs 350, 355, 360, and 365 are shown illustrating features of lymphatic endothelial and muscle cells cocultured in a lymphangion-chip 300. Particularly, graph 350 illustrates gap width in units of μm as a function of time in hours for both LMC monoculture coculture configurations of the lymphangion-chip 300. Graph 355 illustrates LEC density in units of cells per centimeter squared to the fourth ((cells/cm²)×10⁴) as a function of time in hours for both LEC monoculture coculture configurations of the lymphangion-chip 300. Graph 360 illustrates LEC size in units of microns squared (μm²) as a function of time in hours for both LEC monoculture coculture configurations of the lymphangion-chip 300. Finally, graph 365 illustrates LMC density in units of cells per centimeter cubed to the fourth ((cells/cm³)×10⁴) as a function of time in hours for both LMC monoculture coculture configurations of the lymphangion-chip 300.

As indicated by graph 350, the gap between LEC and LMC layers stayed nearly uniform under LMC monoculture, but it reduced steadily over time and reached nearly 5 μm in approximately four days after LEC-LMC coculture. Thus, LMCs respond and migrate toward LECs resulting in a time-dependent decrease in the subendothelial gap that is consistent with prior in vivo and in vitro observations for vascular cells. Correspondingly, observation of on-chip cell culture over four days post-seeding revealed that the LEC density increased over time and reached confluency within three days after culture, as indicated by graph 355. This observation was independent of the presence of LMCs, however, when LECs were cocultured with LMCs, their cell density was significantly lower compared to monoculture conditions. The increase in the LEC density positively correlated with the lower average cell size over time due to the squeezing of cells within the endothelium layer, as indicated in graph 360. Meanwhile, the LMC density also increased in approximately the first two days and plateaued after, with no particularly significant difference between monoculture vs. coculture with LECs at the end of approximately five days, as indicated by graph 365. These on-chip LEC and LMC growth dynamics that we characterized and validated for the lymphatics suggested an active presence of LEC and LMC signaling.

Assessment of Lymphatic Cells Due to Physical Cues within the Lymphangion-Chip

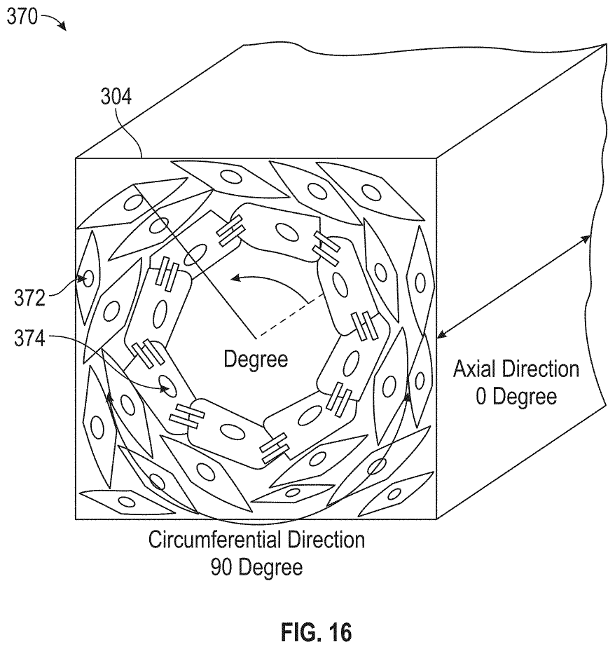

The physiological arrangement of lymphatic cells in vivo is such that a high percentage of muscle and endothelial cells align perpendicular and parallel, respectively, to the vessel's axial direction. This cell alignment—LMCs perpendicular to axially aligned LECs—may be facilitated by the coculture of LECs with LMCs. To test this hypothesis, the lymphatic cell orientation in coculture versus monoculture within the lymphangion-chip 300 was tested. Specifically, three sets of lymphangion-chips 300 were prepared containing only LMCs, only LECs, and an LMC-LEC coculture. After approximately five days of monoculture, the LMCs aligned mostly axially in all lumen sections (sides, top, and bottom). However, cocultured with LECs, most LMCs were circumferentially oriented (i.e., perpendicular to the axial vessel direction). Referring to FIG. 16, a schematic 370 is shown of an embodiment of the microfluidic channel 304 of a lymphangion-chip 300 in which both LMCs 372 and LECs 374 have been cocultured.

When the LEC-LMC coculture was exposed to a typical physiological shear (e.g., 1 dyne/cm²), a significantly greater degree of axial alignment of LECs and circumferential alignment of LMCs was found relative to static culture conditions. Indeed, the LEC alignment in the flow direction within the lymphangion-chip 300 matched the previous in vitro studies for endothelial cells. Additionally, when an intermediate shear (0.1 dyne/cm²) was applied representative of the lymphedema condition in which the lymphatic system's blockage prevents efficient lymph drainage, a relatively poor axial alignment of LECs and circumferential alignment of LMCs was observed.

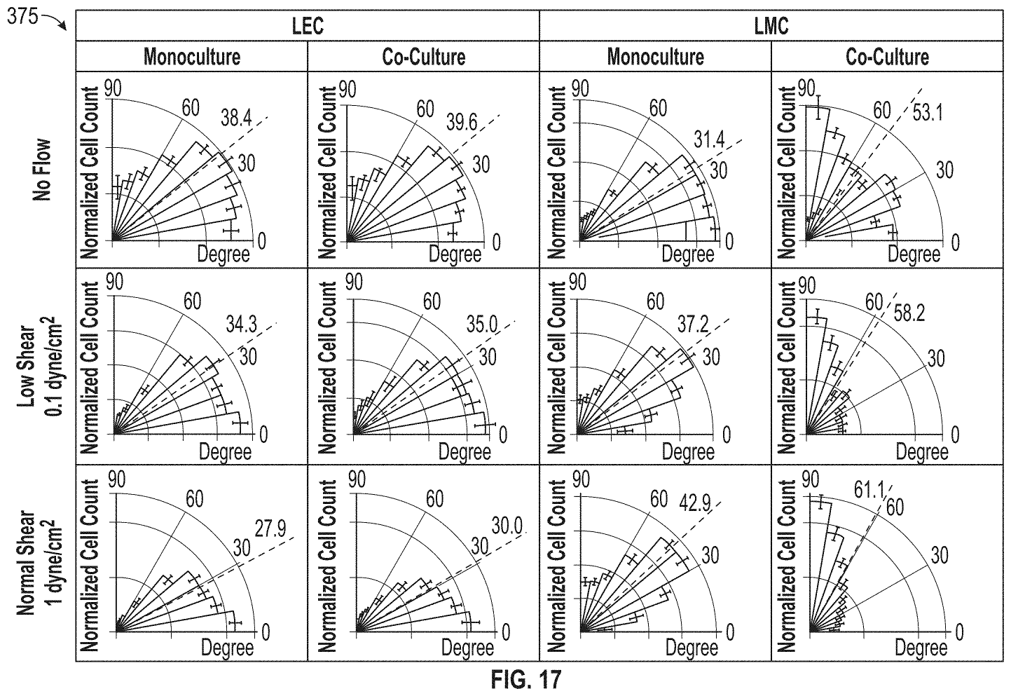

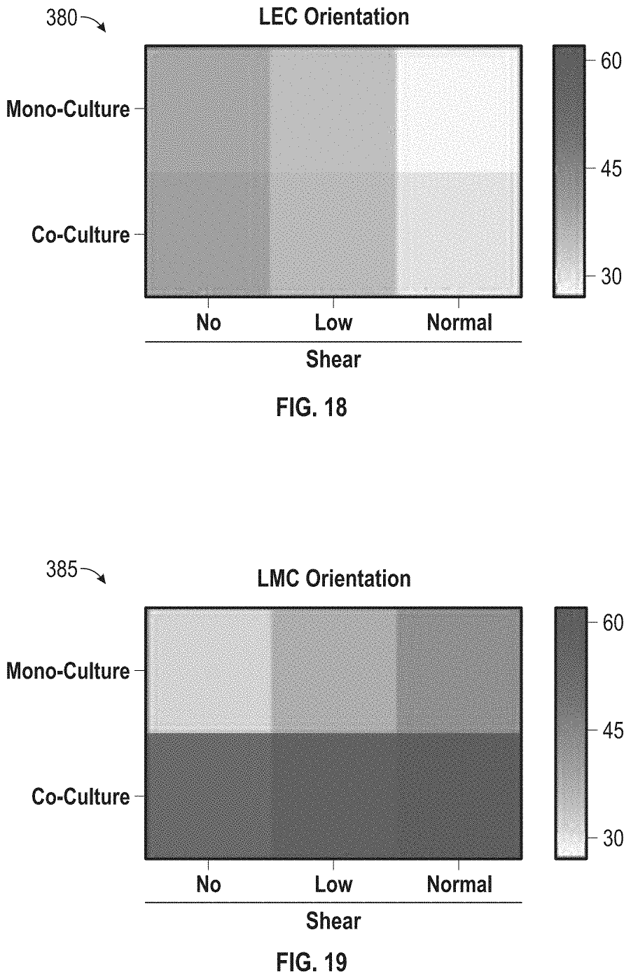

Further, regardless of the magnitude of shear, co-culturing muscle cells with endothelial cells always produced a relatively greater degree of axial orientation of LECs and circumferential orientation of LMCs, strengthening the device's capability to include active signaling between the two cell types. Particularly, referring to FIGS. 17-19, FIG. 17 illustrates a chart 375 indicating the relative orientation of LECs and LMCs under different shear conditions. Additionally, FIG. 18 includes a heatmap 380 indicating LEC mean orientation angle in both monoculture and coculture configurations while being exposed to no flow, low shear conditions, and normal shear conditions. Similarly, FIG. 19 includes a heatmap 385 indicating LMC mean orientation angle in both monoculture and coculture configurations while being exposed to no flow, low shear conditions, and normal shear conditions.

Evaluation of Lymphatic Endothelial Barrier Function Due to Inflammatory Cues within the Lymphangion-Chip

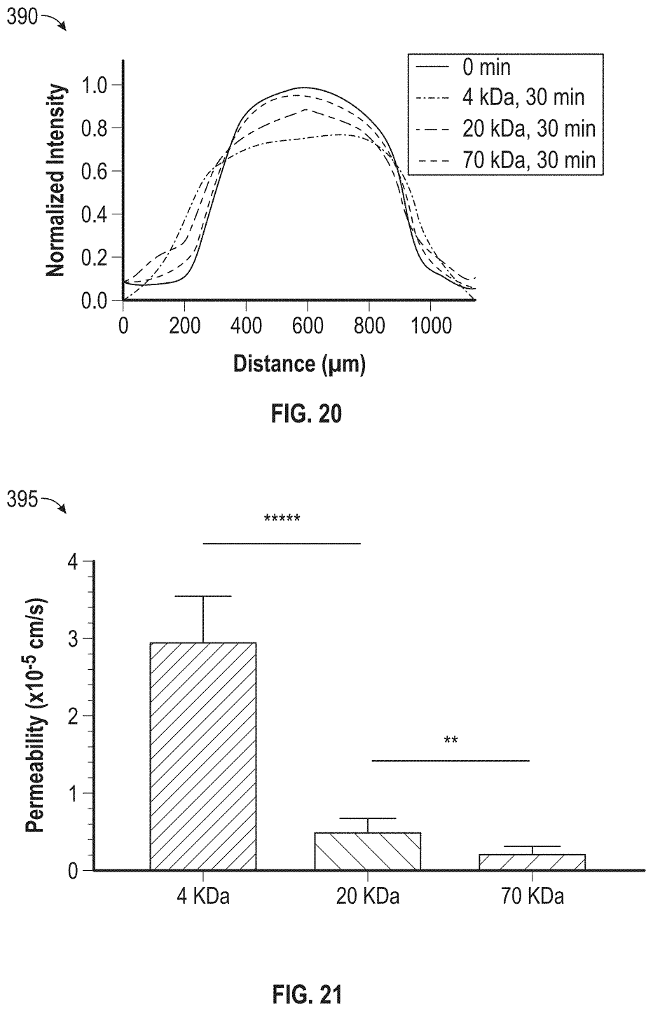

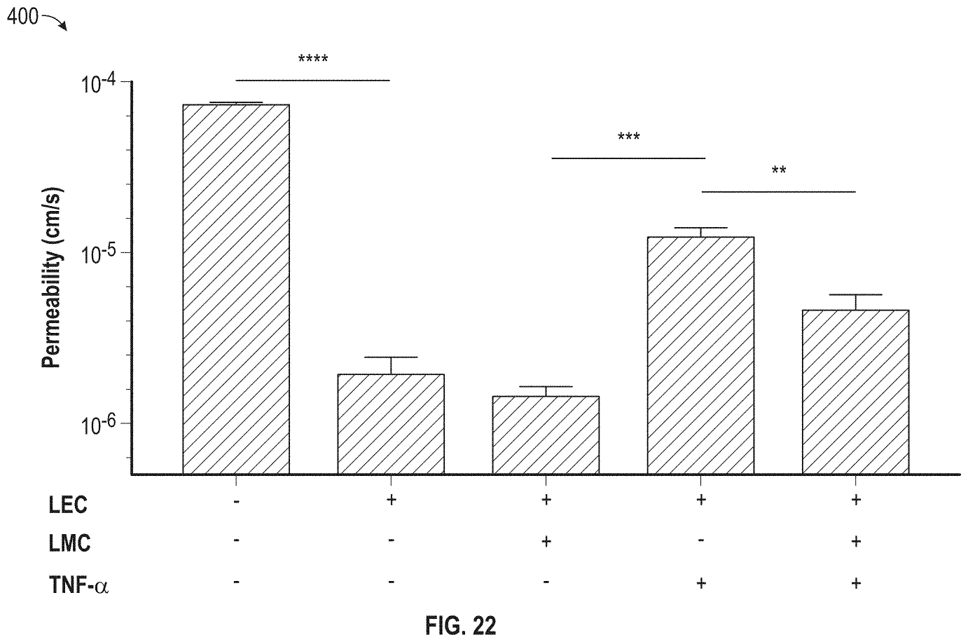

Inflammatory cytokines, including Tumor Necrosis Factor Alpha (TNF-α), may play a role in endothelial dysfunction and increased permeability. TNF-α is known to decrease lymphatic contractility and disrupt the lymphatic endothelial barrier function. Given that the in vitro effect of TNF-α on LEC-LMC coculture has not been characterized before, experiments were conducted to illustrate the power of the lymphangion-chip 300 as a tool to systematically investigate how LMCs could regulate LEC function under the influence of inflammatory signals. First, to characterize the lymphatic endothelial permeability, the diffusion of fluorescein isothiocyanate (FITC)-dextran was measured, resulting in the discovery that dextran may diffuse through lymphatic endothelial cells as a function of its molecular weight. Particularly, referring to FIGS. 20-22, FIG. 20 includes a graph 390 illustrating normalized fluorescence intensity plotted as a function of distance in units of μm for different FITC-conjugate sizes within the lymphangion-chip 300. FIG. 21 includes a graph 395 illustrating measured permeability (in units of centimeters per second to the negative-fifth power ((cm/s)×10⁻⁵)) under the curve for the lymphangion-chip 300. Further, FIG. 22 includes a graph 400 illustrating permeability in units of cm/s for the lymphangion-chip 300 made of only LECs and LEC-LMC coculture before and after exposure to TNF-α as an inflammatory cytokine.

As indicated by graph 395, the vessel permeability for 4 kilodaltons (kDa) molecules (3×10⁻⁵ cm/s) was significantly larger than that for 20 kDa and 70 kDa conjugates (<5×10⁻⁶ cm/s). These results confirm that the lymphangion-chip's 300 endothelium is generally leakier for smaller molecules compared to larger molecules that possess the size of albumin (˜68 kDa), supported by observations made in animal models. Next, when the lymphangion-chip 300 was exposed to TNF-α when LECs and LMCs were cultured alone or together, a significant increase in permeability was found relative to untreated controls. However, when LMCs were cocultured with LECs, we discovered that the lymphatic endothelial barrier function was relatively conserved, suggesting its influence in maintaining tissue homeostasis, as indicated by graph 400.

Discussion

The endothelial and muscle cells are two key cell types that generate and regulate lymph flow in lymphatics and set the vessel's response to mechanical stimulation and inflammation. The normal interactions between these two cell types are important for the homeostasis of the lymphatic transport, and any aberrant interaction between them may lead to loss of junction integrity and flow. However, this LEC-LMC signaling is not fully characterized in experimental models, and the relatively few in vitro studies that currently exist have attempted to unveil the effects of mechanical forces only on LECs. While several vascular organon-chip models have now been published, the muscle layer of these conventional models is not wrapped circumferentially around the endothelium as seen in vivo. Notably, there is currently no design to coculture and study cell signaling between lymphatic endothelial and muscle cells. Importantly, no studies have included the lymphatic muscle cells in their in vitro studies or culture them appropriately with the endothelium.

To address this gap and enable prolonged LEC-LMC coculture in a physiologically relevant environment, the lymphangion-chip 300 was fabricated through gravitational viscous finger patterning or GLP that harnesses the control of the buoyant effect and pressure difference across the channel not accomplished before in conventional models. The results of these experimental studies indicate that the lymphangion-chip 300 provides flexibility in determining the physical and geometrical parameters of a lymphangion. By fabricating the lymphangion-chip 300 with the GLP method, a toolbox is provided to alter the lumen's inner and outer diameter as well as the muscle tissue stiffness and thickness in a robust and physiologically-relevant manner. Therefore, this tunable lymphangion-chip 300 may also be leveraged in studying other types of vascular tissues.

In addition, the observation of a time-dependent decrease in the subendothelial gap strongly suggests proactive LEC-LMC signaling as LECs and LMCs grow and proliferate within the lymphangion-chip 300. The growth factors released by endothelial cells, such as polypeptide platelet-derived growth factor-B (PDGF-B), may produce a concentration gradient around the endothelial layer, promoting the proliferation and migration of LMCs toward the endothelium layer, via their surface receptors, such as tyrosine kinase receptor (PDGFR-B). Also, proliferative smooth muscle cells are known to inhibit endothelial cell proliferation. Within the lymphangion-chip 300, a similar growth pattern was observed, and the LEC growth rate was inhibited under coculture conditions.

Given that the lymphangion-chip 300 produces a symmetrical and cylindrical lumen surrounded by a matrix, LECs and LMCs could be cultured to align in the axial and circumferential direction, respectively, as frequently observed in vivo. The lymphangion-chip 300 further demonstrated a robust sensitivity of this relative alignment of the two cells with respect to the presence or absence of coculture and mechanical forces (shear stress), thus suggesting that LECs and LMCs are biologically and functionally active within the lymphangion-chip 300.

The lymphatic vasculature is essential in modulating the inflammatory response by altering interstitial fluid extravasation and drainage. During inflammation, the lymphatic vessel experiences a significant enlargement in inflamed tissue leading to an elevation in vessel leakiness and thus losing its full functionality. Studying LEC monolayer integrity has shown that endothelium permeability increases in response to inflammatory stimuli. However, the effect of LMCs in cytokine-induced hyperpermeability of the endothelium is largely unknown. The lymphangion-chip 300 revealed the possibility of the contribution of LMCs to partial recovery of endothelial barrier function after exposure to TNF-α. Although the absolute permeability measurements provided herein are typically higher than those quantified for ex vivo animal models, likely due to the difference in methodology, cell type, etc., the trends obtained are consistent with other reports of in vitro lymphatic vascular models.

### CONCLUSION

In summary, the lymphangion-chip 300 allowed for the inclusion of essential lymphatic vascular components in a tunable 3D physiological environment. Particularly, lymphangion-chip 300 allowed for the easy dissection and control of several variables such as flow, geometry, chemical cues, etc., that impact LECs and LMCs in a way that may potentially result in a translational impact. The lymphangion-chip 300 can be immediately combined with molecular and gene analysis tools to provide more precise insight into the regulatory signaling mechanisms of lymphatic vascular physiology/pathophysiology and drug treatments. Finally, the lymphangion-chip 300 can also be applied in blood vascular models.

While embodiments of the disclosure have been shown and described, modifications thereof can be made by one skilled in the art without departing from the scope or teachings herein. The embodiments described herein are exemplary only and are not limiting. Many variations and modifications of the systems, apparatus, and processes described herein are possible and are within the scope of the disclosure. For example, the relative dimensions of various parts, the materials from which the various parts are made, and other parameters can be varied. Accordingly, the scope of protection is not limited to the embodiments described herein, but is only limited by the claims that follow, the scope of which shall include all equivalents of the subject matter of the claims. Unless expressly stated otherwise, the steps in a method claim may be performed in any order. The recitation of identifiers such as (a), (b), (c) or (1), (2), (3) before steps in a method claim are not intended to and do not specify a particular order to the steps, but rather are used to simplify subsequent reference to such steps.

What is claimed is:

1. A method for fabricating an in vitro vessel, the method comprising:
(a) forming a substrate that defines a microfluidic passage therein extending along a longitudinal axis and defined by an inner surface;
(b) positioning the substrate in a vertical orientation whereby an acute angle is formed between the longitudinal axis of the microfluidic passage and the direction of gravity; and
(c) culturing a plurality of first cells in the microfluidic passage while the substrate is disposed in the vertical orientation whereby an annular layer of the plurality of first cells is formed in the microfluidic channel, wherein the layer of the plurality of first cells defines a lumen extending longitudinally through the microfluidic channel.

2. The method of claim 1, wherein the plurality of first cells comprise at least one of lymphatic endothelial cells (LECs), vascular endothelial cells (VECs), lymphatic muscle cells (LMCs), and vascular muscle cells (VMCs).

3. The method of claim 1, further comprising:
(d) culturing a plurality of second cells that are different from plurality of first cells in the microfluidic passage whereby an annular layer of the plurality of second cells is formed in the microfluidic channel.

4. The method of claim 3, wherein the layer of the plurality of first cells forms an annular inner layer of the first cells in the microfluidic channel and the layer of the plurality of second cells forms an outer layer of the plurality of second cells in the microfluidic channel that is radially positioned between the inner layer and the inner surface of the microfluidic channel.

5. The method of claim 3, wherein (d) is performed prior to (b).

6. The method of claim 1, wherein the lumen has an elliptical cross-section.

7. An in vitro vessel comprising:
a substrate forming a microfluidic channel extending along a longitudinal axis and defined by an inner surface, wherein the microfluidic channel extends between a fluid inlet at a first end of the microfluidic channel and a fluid outlet located at a longitudinally opposed second end of the microfluidic channel;
an annular outer layer of muscle cells positioned in the microfluidic channel and extending entirely around the longitudinal axis of the microfluidic channel; and
an annular inner layer of endothelial cells positioned in the outer layer of muscle cells within the microfluidic channel and extending entirely around the longitudinal axis of the microfluidic channel, wherein the inner layer of endothelial cells defines a lumen extending longitudinally through the microfluidic channel and in fluid communication with both the fluid inlet and the fluid outlet formed in the substrate.

8. The in vitro vessel of claim 7, wherein the muscle cells of the outer layer of muscle cells comprise lymphatic muscle cells (LMCs) and the endothelial cells of the inner layer of endothelial cells comprise lymphatic endothelial cells (LECs).

9. The in vitro vessel of claim 7, wherein the muscle cells of the outer layer of muscle cells comprise vascular muscle cells (VMCs) and the endothelial cells of the inner layer of endothelial cells comprise vascular endothelial cells (VECs).

10. The in vitro vessel of claim 7, wherein the outer layer of muscle cells is embedded in an annular extracellular matrix (ECM) positioned in the microfluidic channel and containing collagen.

11. The in vitro vessel of claim 7, wherein a majority of the muscle cells comprising the outer layer of muscle cells are aligned substantially perpendicular to the longitudinal axis of the microfluidic channel.

12. The in vitro vessel of claim 7, wherein a majority of the endothelial cells comprising the inner layer of endothelial cells are aligned substantially parallel to the longitudinal axis of the microfluidic channel.

13. The in vitro vessel of claim 7, wherein a majority of the endothelial cells comprising the inner layer of endothelial cells are aligned substantially perpendicular to a majority of the muscle cells comprising the outer layer of muscle cells.

14. The in vitro vessel of claim 7, wherein the lumen has an elliptical cross-section.

15. An in vitro vessel comprising:
a substrate forming a microfluidic channel extending along a longitudinal axis and defined by an inner surface, wherein the microfluidic channel extends between a fluid inlet at a first end of the microfluidic channel and a fluid outlet located at a longitudinally opposed second end of the microfluidic channel; and
an annular layer of endothelial cells positioned within the microfluidic channel and extending entirely around the longitudinal axis of the microfluidic channel, wherein the layer of endothelial cells defines a lumen having an elliptical cross-section and extending longitudinally through the microfluidic channel and in fluid communication with both the fluid inlet and the fluid outlet formed in the substrate.

16. The in vitro vessel of claim 15, wherein the endothelial cells of the layer of endothelial cells comprise lymphatic endothelial cells (LECs).

17. The in vitro vessel of claim 15, wherein the endothelial cells of the layer of endothelial cells comprise vascular endothelial cells (VECs).

18. The in vitro vessel of claim 15, wherein the elliptical cross-section of the lumen is defined by a major axis and a minor axis extending orthogonal to the major axis of the elliptical cross-section of the lumen, and wherein a ratio of the major axis to the minor axis is between 1.1:1 and 5:1.

19. The in vitro vessel of claim 18, wherein the elliptical cross-section of the lumen is defined by a major axis and a minor axis extending orthogonal to the major axis of the elliptical cross-section of the lumen, and wherein the ratio of the major axis to the minor axis is between 1:1 and 3:1.

20. The in vitro vessel of claim 18, wherein a ratio of the minimum radial thickness of the layer of endothelial cells to the maximum radial thickness of the layer of endothelial cells is between 0.5:1 and 2:1.

21. The in vitro vessel of claim 18, wherein:
the layer of endothelial cells positioned within the microfluidic channel defines an annular inner layer of endothelial cells; and
an annular outer layer of muscle cells positioned in the microfluidic channel radially between the inner layer of endothelial cells and the inner surface of the microfluidic channel, and wherein the outer layer of muscle cells extends entirely around the longitudinal axis of the microfluidic channel.

22. The in vitro vessel of claim 21, wherein a ratio of the minimum radial thickness of the layer of muscle cells to the maximum radial thickness of the layer of muscle cells is between 0.5:1 and 2:1.
